(19) United States
(12) Patent Application Publication
(10) Pub. No.: US 2005/0191341 A1
(43) Pub. Date: Sep. 1, 2005

(54) Liposomal composition comprising L-theanine

(76) Inventor: Gottfried Kellermann, Osceola, WI (US)

Correspondence Address: DOUGHERTY, CLEMENTS & HOFER, 1901 ROXBOROUGH ROAD, SUITE300, CHARLOTTE, NC, 28211, US

(21) Appl. No.: 10/790,619

(22) Filed: Mar. 1, 2004

(51) Int. Cl. A61K 9/127; A61K 31/405; A61K 31/198

(52) U.S. Cl. 424/450; 514/419; 514/561

(57) ABSTRACT

A liposomal encapsulated solution of L-theanine is sprayed onto the sublingual membrane of a subject. The method may be used in the treatment of stress, tension headaches, attention deficit disorder (ADD), attention deficit disorder hyperactivity (ADHD), claustrophobia, hyperactivity, and anxiety.

### BACKGROUND OF THE INVENTION

The present invention relates to a liposomal composition comprising theanine and to a method of treating tension headaches and other ailments in humans and animals using the composition. A preferred composition of the invention also comprises 5-hydroxytryptophan.

It is desirable to treat unwanted conditions and ailments by using effective compositions made of conventional ingredients which have a history of use without serious adverse side effects or adverse consequences. It is particularly desirable to employ naturally occurring materials which have a long history of use so that we have a long time experience concerning their effects.

Neurotransmitters and many of the compounds useful in their manipulation are naturally occurring materials with which we have a long time experience. In recent years, there has been considerable research relating to neurotransmitters, the functions of neurotransmitters, methods of manipulation of neurotransmitters, and the effect of manipulation of neurotransmitters on various disorders. Neurotransmitters are believed to be involved in many disorders relating to the nervous system such as attention deficit disorder (ADD), attention deficit disorder hyperactivity (ADHD) in children, tension headaches, claustrophobia, hyperactivity, stress, and anxiety. There remains a need, however, for further research and development in the field of neurotransmitters and their manipulation for treatment of disorders.

L-theanine is not itself a neurotransmitter but is an amino acid known to induce relaxation and lower stress. Green tea and other compositions containing L-theanine have been marketed for many years with advertising touting their beneficial effects to induce relaxation and lower stress levels. The ability of L-theanine to reduce the stimulatory effects of caffeine and to increase the effectiveness of some anti-tumor drugs is also known.

L-theanine is known to reduce excitatory neurotransmission by modulating the excretion of neurotransmitters like serotonin and dopamine, as well as affecting the transport of glutamate to down regulate its function which, in turn, enhances GABA (gamma-amino butyric acid) function. GABA is the primary inhibitory neurotransmitter. Glutamate is the primary excitatory neurotransmitter. L-theanine is a neurotransmitter modulator which depresses dopamine and serotonin levels but is not believed to have any direct therapeutic effect, only an indirect and symptomatic calming effect. Therefore, it is believed to be preferable to administer L-theanine in an amount which is the least amount required to be effective to reduce or eliminate undesired symptoms.

In accordance with the present invention, it has been discovered that L-theanine can be delivered or administered to a human or other animal in the form of a liposomal spray solution with focused and effective results. The use of a liposomal spray sublingual delivery system quickly delivers an effective amount of L-theanine yet requires a relatively small amount of L-theanine to be effective. It has been found that oral administration of a liposomal composition comprising L-theanine has a profound effect on certain neurotransmitter related disorders even when delivered in surprisingly low concentrations.

While delivery of drugs and nutrients to humans and animals by liposomal delivery systems is known, most liposomal delivery schemes rely on uptake in and post the stomach. Oral administration using liposomal delivery systems through the sublingual mucosal membranes is less often used and not known for use to deliver L-theanine. Thus, in accordance with the present invention a novel combination has been discovered which obtains surprisingly effective results.

While L-theanine is used for its symptomatic effect, the administration of an effecive amount of L-theanine reduces neurotransmitter levels. Unfortunately, low levels of the neurotransmitter seratonin are common in the population, and where a patient is suffering from stress, typically there is also a reduction of seratonin level. It has been found that the additional use of 5-hydroxytryptophan (5-HTP), another inhibitory neurotransmitter, in the present invention further enhances the symptomatic effect of L-theanine while increasing neurotransmitter levels. Thus, in a preferred embodiment of the present invention, an improved delivery system comprises L-theanine and 5-HTP in a liposomal solution. The present invention also provides an oral spray method of using the improved delivery system for treatment of stress, tension headaches, attention deficit disorder (ADD), attention deficit disorder hyperactivity (ADHD), claustrophobia, hyperactivity, and anxiety. Further understanding of the composition and methods of the present invention will be had from the following specification taken in conjunction with the claims appended hereto.

### SUMMARY OF THE INVENTION

A treatment composition of the present invention is a liposomal encapsulated solution of L-theanine. The treatment composition is preferably administered to a subject by oral spraying an effective amount of the composition onto the sublingual membrane. The method may be used in the treatment of stress, tension headaches, attention deficit disorder (ADD), attention deficit disorder hyperactivity (ADHD), claustrophobia, hyperactivity, and anxiety.

A preferred composition of the present invention comprises, in addition to L-theanine, an effective amount of 5-hydroxytryptophan (5-HTP). The treatment composition is effective to address neurotransmitter over-stimulation and can be used in young patients with high amine transmitter turnover, high amine transmitter excretion, or increased GABA excretion. The treatment composition administered in accordance with the method of the present invention provides an unexpected result of quickly reducing high neurotransmitter excretion, not only confirming observations that theanine effects dopamine and serotonin, but also showing that theanine effects epinephrine, norepinephrine, and PEA (phenylethylamine)

### DETAILED DESCRIPTION OF THE INVENTION

A composition of the present invention is a liposomal encapsulated solution of theanine. More specifically, a composition of the present invention comprises an aqueous solution of L-theanine encapsulated by liposomes. In a preferred embodiment, the liposomal composition comprises, in addition to L-theanine, 5-hydroxytryptophan (5-HTP). Theanine and 5-HTP are present in the composition in an amount or concentration effective to achieve the desired therapeutic effect.

Theanine is an amino acid that is contained in tea leaves and is a glutamic acid derivative. Theanine has been used for many years as a food additive for seasoning. It may be prepared by several different methods such as those disclosed in U.S. Pat. No. 6,589,566 B2 Jul. 8, 2003 to Ueda et al, the disclosure of which is specifically incorporated by reference herein. Theanine is available commercially, for example, as Suntheanine® from Taiyo Kagaku Co., Ltd, Akahorishinmachi, Yokkaichi-shi, Mie, 510-0825, Japan. The L-form of theanine is preferred in the present invention because it is approved as a food additive.

The composition of the present invention preferably comprises 5-hydroxytryptophan (5-HTP) in addition to enhance the effect of the theanine component and to maintain neurotransmitter balance.

It will also be appreciated that a number of other components regulate the balance of excitatory and inhibitory transmitters and such other modulators of this balance may be included in the present composition. These include other amino acids such as taurine, glutamine, GABA, phenylalanine, tyrosine, dopa, glutamine, glutamate, SAMe and cysteine. Such other components may be vitamin and mineral components that address neurotransmitter synthesis, release, or function such as magnesium, calcium, chromium, selenium, folic acid, riboflavin, pantothenic acid, vitamin B6, B12, and C.

Liposomes for use in making the liposomal composition of the present invention are those liposomes which are nutritionally safe and are able to encapsulate the aqueous theanine solution. Liposomes are composed of lipid bilayers which form closed shells surrounding an internal liquid phase.

In making the present composition, an aqueous solution of theanine is shaken or otherwise caused to mix with a liposome component which encapsulates the theanine solution. Separate solutions of L-theanine and 5-HTP can also be mixed with a suitable liposome component to form the preferred composition. The exact amount of L-theanine and/or 5-HTP in the solution is generally not critical so long as an effective amount of L-theanine and/or 5-HTP is available for administration to the patient. The mixture is then packaged in a spray bottle for subsequent administration use by the end user.

In use and in accordance with the treatment method of the present invention, the mixture, or treatment composition, is administered to a subject by oral spray, preferably directly onto the sublingual membrane. The liposomal encapsulated theanine or the liposmal encapsulated theanine and 5-HTP, is transported through the sublingual mucosal membrane directly and quickly into the blood stream. Delivery of L-theanine or L-theanine and 5-HTP, to operative sites is surprisingly fast. Hence, the amount of theanine, or the amount of theanine and 5-HTP, required to be effective is surprisingly small.

The treatment method of this invention can be used to treat or ameliorate various symptoms such as tension headaches, stress, attention deficit disorder (ADD), attention deficit hyperactivity disorder (ADHD), claustrophobia, hyperactivity, and anxiety. The method has been found to be particularly useful in treating tension headaches. However, the method can be used to treat any problem related to neurotransmitter over-stimulation. The method quickly reduces high neurotransmitter excretion and is useful to treat young patients with high amine transmitter turnover, amine transmitter excretion, or increased GABA excretion. The preparation of liposomes as a delivery system is well known and a number of techniques may be employed such as those detailed in Szoka, F., Jr., et al., Ann. Rev. Biophys. Bioeng. 9:467 (1980). Also, suitable techniques are disclosed in U.S. Pat. No. 6,316,024 Nov. 13, 2001, to Allen et al. for “Therapeutic Liposome Composition and Method of Preparation” the disclosure of which is specifically incorporated by reference herein. Suitable liposomes are commercially available from, for example, BioZone Laboratories, Inc., 580 Garcia Avenue, Pittsburg, Calif., 94565, USA

Typically, liposomes are multilamellar vesicles (MLVs), which can be formed by simple lipid-film hydration techniques. In this procedure, a mixture of liposome-forming lipids dissolved in a suitable organic solvent is evaporated in a vessel to form a thin film, which is then covered by an aqueous medium. The lipid film hydrates to form MLVs, typically with sizes between about 0.1 to 10 microns.

Liposomes may also comprise a vesicle-forming lipid derivatized with a hydrophilic polymer to form a surface coating of hydrophilic polymer chains on the liposome's surface. Such a coating is preferably prepared by including between 1-20 mole percent of the derivatized lipid with the remaining liposome forming components, e.g., vesicle-forming lipids. Exemplary methods of preparing derivatized lipids and of forming polymer-coated liposomes are described in U.S. Pat. No. 5,013,556 May 7, 1991 to Woodle et al. for “Liposomes with Enhanced Circulation Time”; U.S. Pat. No. 5,631,018 May 20, 1997 to Zalipsky et al. for “Lipid-polymer Conjugates and Liposomes” and U.S. Pat. No. 5,395,619, May 7, 1995 to “Zalipsky et al. for “Lipid-polymer Conjugates and Liposomes” each of which patents are specifically incorporated herein by reference. It will be appreciated that the hydrophilic polymer may be stably coupled to the lipid, or coupled through an unstable linkage which allows the coated liposomes to shed the coating of polymer chains as they circulate in the bloodstream or in response to a stimulus.

The L-theanine and/or 5-HTP can be incorporated into liposomal solutions by standard methods, including (i) passive entrapment of a water-soluble compound by hydrating a lipid film with an aqueous solution of the agent, (ii) passive entrapment of a lipophilic compound by hydrating a lipid film containing the agent, and (iii) loading an ionizable drug against an inside/outside liposome pH gradient. Other methods, such as reverse evaporation phase liposome preparation, are also suitable.

In a preferred method, the liposomes are prepared as taught in U.S. Pat. No. 6,387,397 May 14, 2000 to Chen et al for “Polymerized Liposomes Targeted to M Cells and Useful for Oral or Mucosal Drug Delivery” which patent is specifically incorporated by reference herein. The polymerized liposomes of the present invention may be prepared by a variety of techniques. For example, polymerized liposomes can be prepared by polymerizing double and triple bond-containing olefinic and acetylenic phospholipids. Alternatively, polymerized liposomes can be prepared by chemical oxidation of thiol groups in the phospholipids to disulfide linkages. The polymerization can take place in a solution containing a substance such as L-theanine and/or 5-HTP, or a drug or antigen, in which case the substance is encapsulated during the polymerization. Alternatively, the liposomes can be polymerized first, and L-theanine and/or 5-HTP can be added later by resuspending the polymerized liposomes in a solution of L-theanine and/or 5-HTP and entrapping the L-theanine and/or 5HTP by sonification of the suspension. Another method of entrapping L-theanine and/or 5-HTP in polymerized liposomes is to dry the polymerized liposomes to form a film, and hydrate the film in a solution of the L-theanine and/or 5-HTP. The above conditions should, of course, be mild enough to entrap L-theanine and/or 5-HTP without damage.

Polymerized liposomes are generally prepared by polymerization of double bond-containing monomeric phospholipids. These phospholipids may contain any unsaturated functional group, including polymerizable functional group double or triple bonds, any may contain more than one polymerizable functional group double or triple bonded. Suitable monomeric phospholipids are known to those skilled in the art, and include, but are not limited to, phosphatidylaholines DODPC (1,2-di(2,4-Octadecadienoyl)-3-phosphatidylcholine), 2,4-diene phospholipids, di-yne phospholipids, see e.g., U.S. Pat. No. 4,485,045, Nov. 27, 1984 to Regen for “Synthetic Phosphatidyl Cholines Useful in Forming Liposomes” and U.S. Pat. No. 4,861,521 Aug. 29, 1989 to Suzuki et al. for “Polymerizable Liposome-forming Lipid, and Method for Production” each of which patents are specifically incorporated by reference herein. If the liposome is polymerized by oxidation of thiol groups, it is preferred not to encapsulate thiol-containing biologically active substances, as they could be oxidized during the polymerization step.

The liposomes of the present invention may be polymerized by free radical initiation. The monomeric double bond-containing phospholipids can be polymerized using a hydrophobic free radical initiator, such as AIBN (azo-bis-isobutyronitrile), or a hydrophilic free radical initiator such as AAPD (azo-bis-amidinopropane dihydrochloride). The latter is particularly useful for initiating polymerization between layers of the bilayer. The present invention also encompasses the use of other mild redox initiators, such as Na₂ S₂ O₅ and K₂ S₂ O₈. Alternatively, polymerization can be initiated by using a radiation source, such as ultraviolet or gamma radiation. Use of the free radical initiators is preferred if the biologically active substance to be entrapped is denatured when exposed to radiation.

The ratio between the phospholipid and crosslinker and aqueous phase all affect the percent of crosslinking. In general, the percent crosslinking increases as the amount of crosslinker or time or temperature of reaction are increased. As the percent crosslinking increases, the release rate of the materials from the liposomes decreases and the stability increases.

Any subject can be administered the compositions of the present invention. Such subjects include humans as well as other mammalian, avian, reptilian, or amphibian organisms. Agents, such as nutritional supplements, are becoming components in the diets of domesticated mammals such as pets and livestock.

When administered, the compositions of the present inventions optimally are held in the oral cavity for a period of time sufficient for absorption of the agent sublingually. The period of time needed to obtain absorption will vary based mainly on the type of lipid particle, the lipids that make up the lipid coat, the agent encapsulated, and delivery system used. A skilled artisan can readily determine the time needed to achieve, e.g., sublingual absorption and vary these parameters to optimize delivery for a given product.

The following examples are intended to illustrate, but not limit, the present invention.

### EXAMPLE 1

A liposomal composition is prepared by following the methodologies disclosed in U.S. Pat. No. 5,891,465. The composition comprises:

Liposomal Theanine Spray Composition A

| Ingredients | % w/w |
|---|---|
| Purified Lecithin (Phospholipon 90) | 2.00 |
| Cholesterol | 0.20 |
| Tocopherol Acetate | 0.40 |
| Theanine | 7.14 |
| Pyridoxine HCL | 0.05 |
| Glycerin | 7.50 |
| Ethyl Alcohol | 1.00 |
| Sodium Benzoate | 0.15 |
| Polysorbate 20 | 1.00 |
| Flavor | 1.00 |
| Citric Acid | 0.15 |
| Spevia/nat. sweet | 0.25 |
| Citrus seed extract | 0.05 |
| Purified Water, USP, | qs. ad. |

The composition was prepared by commingling ingredients lecithin, ethyl alcohol, tocopherol acetate, cholesterol and glycerin (“the ethyl alcohol mixture”) in a large volume flask. In a separate beaker, water, theanine, pyridoxine, sodium benzoate, polysorbate 20, and citric acid (“the water mixture”) were mixed and heated to 50° C. The water mixture was added to the ethyl alcohol mixture while vigorously mixing with a high speed, high shear homogenizing mixer at 750-1500 rpm for 30 minutes.

The homogenizer was stopped and the solution was placed on a magnetic plate, covered with parafilm and mixed with a magnetic stir bar until cooled to room temperature. Flavor and citrus seed extract were added and the solution was placed in an appropriate spray dispenser.

Analysis of the preparation under an optical light microscope with polarized light at 400 X magnification confirmed the presence of both multilamellar lipid vesicles (MLV) and unilamellar lipid vesicles.

### EXAMPLE 2

A liposomal composition was prepared of:

Liposomal Theanine & 5-HTP Spray Composition B

| Ingredients | % w/w |
|---|---|
| Purified Lecithin (Phospholipon 90) | 2.00 |
| Cholesterol | 0.20 |
| Tocopherol Acetate | 0.40 |
| Theanine | 7.14 |
| 5-HTP | 2.14 |
| Pyridoxine HCL | 0.05 |
| Glycerin | 7.50 |
| Ethyl Alcohol | 1.00 |
| Sodium Benzoate | 0.15 |
| Polysorbate 20 | 1.00 |
| Flavor | 1.00 |
| Citric Acid | 0.15 |
| Spevia/nat. sweet | 0.25 |
| Citrus seed extract | 0.05 |
| Purified Water, USP, | qs. ad. |

The liposomal composition was prepared by following the procedure of Example 1 except that 5-HTP was added to and mixed with the ethyl alcohol mixture and the mix was heated to 55° C.

### EXAMPLE 3

A liposomal compositon was prepared of:

| Ingredients | % w/w |
|---|---|
| Purified Lecithin (Phospholipon 90) | 2.00 |
| Cholesterol | 0.20 |
| Tocopherol Acetate | 0.40 |
| Theanine | 7.14 |
| 5-HTP | 2.14 |
| Pyridoxine HCL | 0.50 |
| Folic acid | 0.00714 |
| Glycerin | 7.50 |
| Ethyl Alcohol | 1.00 |
| Sodium Benzoate | 0.15 |
| Polysorbate 20 | 1.00 |
| Flavor | 1.00 |
| Citric Acid | 0.15 |
| Spevia/nat. sweet | 0.25 |
| Citrus seed extract | 0.05 |
| Purified Water, USP, | qs. ad. |

The liposomal composition was prepared by following the procedure of Example 2 except that Vitamin B6 and Folic acid are also added to and mixed with the water mixture.

### EXAMPLE 4

A subject having symptoms of tension headache, anxiety, and elevated heart rate, was treated by administering the composition of Example 1 to the subject by spraying 1.4 ml of a 7% theanine liposomal composition (100 mg theanine), the composition of Example 1, into the mouth of the subject. A study was then conducted to evaluate the effects of the composition on the levels of neurotransmitters.

The following neurotransmitter levels were measured at the indicated times:

Urinary neurotransmitter levels in ug/gCr

| | | | | | | PEA |
|---|---|---|---|---|---|---|
| | Epinephrine | Norepinephrine | Dopamine | Serotonin | GABA | (Phenylethylamine) |
| Before | 21.3 | 74.2 | 227.4 | 433.9 | 8.2 | 1814.3 |
| Treatment | | | | | | |
| 3 hours | 8.7 | 29.5 | 109.0 | 176.6 | 4.4 | 790.6 |
| post | | | | | | |
| treatment | | | | | | |

GABA refers to gamma-amino butyric acid and PEA refers to phenylethylamine.

Three hours after oral treatment with a 7% L-theanine liposomal composition prepared as described in Example 1, the subject's symptoms of tension headache were alleviated, anxiety was significantly reduced, and heart rate returned to normal. The laboratory data support the changes in the clinical observations of alleviated headache and reduced anxiety, since epinephrine, norepinephrine, and dopamine are reduced from elevated to normal values. GABA and PEA levels have also been reduced.

### EXAMPLE 5

A subject having symptoms of tension headache, anxiety, and stress, was treated two times per day, once in the morning and once in the evening, for one week by administering the composition of Example 1. Each administration is given by spraying 1.4 ml (100 mg theanine) of the composition into the mouth of the subject.

The following neurotransmitter levels were measured at the indicated times:

| | Epinephrine | Norepinephrine | Dopamine | Serotonin |
|---|---|---|---|---|
| Before | 29.6 | 52.6 | 178.8 | 232.8 |
| Treatment | | | | |
| 8 hours | 4.9 | 59.7 | 111.7 | 98.7 |
| post | | | | |
| treatment | | | | |

Eight hours after oral treatment with the 7% L-theanine liposomal composition prepared as described in Example 1, the subject's symptoms of tension headache and anxiety were alleviated. After the treatment period, the subject reported feeling much less stressed and more able to relax. The laboratory data support the changes in the clinical observations of alleviated headache and anxiety. Epinephrine and dopamine are reduced and the serotonin secretion—a sign of chronic stress—has diminished.

### EXAMPLE 6

A subject having symptoms of recurrent headache, stress, and an inability to relax, is treated twice per day, once in the morning and once in the evening, by administering the composition of Example 1 to the subject by spraying 1.4 ml (100 mg theanine) of the composition into the mouth of the subject BID.

The following neurotransmitter levels were measured at the indicated times:

Urinary neurotransmitter levels in ug/gCr

| | Before Treatment | One week of ongoing treatment |
|---|---|---|
| Epinephrine | 7.4 | 3.7 |
| Norepinephrine | 49.8 | 53.1 |
| Dopamine | 297.8 | 110.8 |
| Serotonin | 87.2 | 79.8 |
| GABA | 7.53 | 2.0 |
| PEA | 693.3 | 260.5 |
| Histamine | 26.5 | 28.8 |

After one week of oral treatment BID with a 7% L-theanine liposomal composition prepared as described in Example 1, the patient reported an increased ability to relax, significantly reduced severity of headaches, and improved sleep. The laboratory data support the changes in the clinical observations, as the stimulatory presentation and the stimulatory neurotransmitters, epinephrine, dopamine, and PEA, are both reduced. GABA levels have also been reduced indicating there is no longer an imbalance in the levels of the excitatory and inhibitory neurotransmitters.

1. A liposomal composition comprising a liposomal encapsulated aqueous solution of an effective amount of theanine.

2. The composition of claim 1 wherein said theanine is L-theanine.

3. The composition of claim 1 wherein said composition comprises an effective amount of 5-Hydroxytryptophan.

4. The composition of claim 1 wherein said theanine is present in a concentration from about 1% to about 20%.

5. The composition of claim 4 wherein said theanine is present in a concentration from about 2% to about 10%.

6. The composition of claim 2 wherein said composition comprises, in addition to said theanine and 5-Hydroxytryptophan, an additional therapeutic moiety.

7. A method of treating a subject, said method comprising sublingually administering to said subject an effective amount of a liposomal encapsulated aqueous solution of theanine.

8. The method of claim 7 wherein said theanine is L-theanine.

9. The method of claim 8 wherein said theanine is present in said composition in a concentration from about 1% to about 20%.

10. The method of claim 9 wherein said theanine is present in said composition in a concentration from about 2% to about 10%.

11. The method of claim 7 wherein said composition comprises, in addition to theanine, an effective amount of 5-Hydroxytryptophan.

12. The method of treating a subject having neurotransmitter over-stimulation by sublingually administering an effective amount of a liposomal encapsulated aqueous solution of L-theanine to said subject.

13. A spray bottle containing a liposomal encapsulated aqueous solution of theanine.
